(19) United States
(12) Patent Application Publication
(10) Pub. No.: US 2026/0041741 A1
(43) Pub. Date: Feb. 12, 2026

(54) CD39 AND CD73 FOR THE THERAPY OF HEMORRHAGIC SHOCK

(71) Applicant: The Trustees of Columbia University in the City of New York, New York, NY (US)

(72) Inventors: George Hasko, Ramsey, NJ (US); Taha Kelestemur, Fort Lee, NJ (US)

(21) Appl. No.: 19/363,111

(22) Filed: Oct. 20, 2025

Related U.S. Application Data
(63) Continuation of application No. PCT/US2024/025943, filed on Apr. 24, 2024.
(60) Provisional application No. 63/461,647, filed on Apr. 25, 2023.

(51) Int. Cl. A61K 38/46; A61P 9/14

(52) U.S. Cl. CPC A61K 38/46; A61K 38/465; A61P 9/14; C12Y 301/03005; C12Y 306/01005

(57) ABSTRACT

Compositions and methods for treating hemorrhagic shock, multiple organ failure resulting from hemorrhagic shock, and organ complications resultant therefrom such as acute respiratory distress syndrome.

### CROSS-REFERENCE TO RELATED APPLICATIONS

This application is a continuation of PCT International Application No. PCT/US2024/025943, filed Apr. 24, 2024, which claims benefit of U.S. Provisional Application No. 63/461,647, filed Apr. 25, 2023, the contents of each of which are hereby incorporated by reference.

### STATEMENT OF GOVERNMENTAL INTEREST

This invention was made with government support under HL158519 and GM066189 awarded by the National Institutes of Health. The government has certain rights in the invention.

### BACKGROUND

The disclosures of all publications, patents, patent application publications and books referred to in this application are hereby incorporated by reference in their entirety into the subject application to more fully describe the art to which the subject invention pertains.

Death from hemorrhagic shock (HS) represents a substantial global problem, with an estimated 1.9 million deaths per year worldwide, 1.5 million of which result from physical trauma. Furthermore, those who survive the initial hemorrhagic insult have poor functional outcomes and significantly increased short- or long-term morbidity and mortality. Trauma patients who survive the early post-injury period can develop a wide range of conditions, such as acute lung injury, acute respiratory distress syndrome, systemic inflammatory response syndrome, multiple organ dysfunction syndrome, multiple organ failure, and sepsis. Multiple organ failure following trauma, HS and resuscitation is a consequence of a systemic ischemia-reperfusion injury, which leads to systemic inflammation, cellular damage and immune dysfunction.

### SUMMARY

Herein are disclosed compositions and methods for treating hemorrhagic shock, multiple organ failure resulting from hemorrhagic shock, and organ complications resultant therefrom such as acute respiratory distress syndrome.

A method of treating hemorrhagic shock or hypovolemic shock in a subject, of reducing the chance of multi-organ failure in a subject who has had a hemorrhagic shock or hypovolemic shock, or for ameliorating ischemia-reperfusion injury in an organ of a subject who has had a hemorrhagic shock or hypovolemic shock, the method comprising administering to the subject an amount of a CD39 mimic and/or CD73 mimic effective to treat hemorrhagic shock or hypovolemic shock.

A method for treating or reducing development of acute respiratory distress syndrome in a subject, comprising administering to the subject an amount of a CD39 mimic and/or CD73 mimic effective to treat or reduce development of acute respiratory distress syndrome.

A method comprising:
diagnosing a subject as having hemorrhagic shock or hypovolemic shock, and optionally diagnosing the patient as not having an infection or as not having a non-sterile shock;
administering to the subject so-diagnosed an amount of a CD39 mimic and/or CD73 mimic effective to reduce or treat an effect of hemorrhagic shock or hypovolemic shock.

A method of treating hypovolemic shock in a subject comprising transfusing into the subject having hypovolemic shock an amount of a blood product or blood replacement product which comprises an amount of a CD39 mimic and/or CD73 mimic, sufficient to treat hypovolemic shock.

A composition for treating hemorrhagic shock or hypovolemic shock, or for ameliorating ischemia-reperfusion injury in an organ, comprising:
a blood product or blood replacement product; and
an amount of a CD39 mimic and/or CD73 mimic.

A method of decreasing liver injury, or reducing an increase in lung permeability and neutrophil infiltration, associated with hypovolemic shock in a subject comprising transfusing into the subject having hypovolemic shock an amount of a CD39 mimic and/or CD73 mimic, sufficient to decrease liver injury, or sufficient to decrease an increase in lung permeability and neutrophil infiltration, associated with hypovolemic shock.

### BRIEF DESCRIPTION OF THE DRAWINGS

FIG. 1: Experimental design. Mice are exposed to T/SS or T/HS for 2.5 h. Mice received vehicle, agonist, or antagonist 30 min before shock induction. After a 15-min resuscitation period and a subsequent observation period of 3 hours, mice were euthanized and tissues were collected.

FIGS. 2A-2C: T/HS increases CD39, CD73, and A2BAR expression in lung, liver, kidney, and gut. (A, B, C). CD39, CD73, and A2BAR expression were evaluated using western blotting of protein extracts. Data are mean±S.D. (n=4/group). ** p<0.01 compared with T/SS.

FIGS. 3A-3C: ATP (A), adenosine (B), and cAMP (C) levels in BALF following T/HS in the absence of CD39, CD73, and A2BAR samples. ATP and CAMP were measured colorimetrically and adenosine was measured by a fluorometric assay. Data are mean±S.D. (n=4/group) *p<0.05 compared with T/SS-V, ** p<0.01 compared with T/SS-V, #p<0.05 compared with T/HS-V, ##p<0.01 compared with T/HS-V.

FIGS. 4A-4H: CD39, CD73, and A2BR regulation of lung permeability and MPO activity. Lung permeability was determined using the EBD method (A, C, E, G) and MPO activity as a surrogate for neutrophil sequestration (B, D, F, H) was determined spectrophotometrically. Data are mean±S.D. (n=4/group). * p<0.05 compared with corresponding T/SS, ** p<0.01 compared with corresponding T/SS, #p<0.05 compared with corresponding T/HS-V, ##p<0.01 compared with corresponding T/HS.

FIGS. 5A-5B: Effect of NECA on lung MPO activity in CD39−/− and CD73−/− mice. MPO activity was determined spectrophotometrically (A, B). Data are mean±S.D. (n=4/group). *p<0.05 compared with T/HS, ** p<0.05 compared with T/HS.

FIGS. 6A-6C: Quantification of pro- and anti-inflammatory cytokines and chemokines in the lung. Representative images of cytokine-chemokine arrays that were used to interrogate cytokine expression (A). Proteome Profiler Mouse Cytokine-Chemokine array was used to analyze cytokines in mice subjected to T/HS. Data were visualized by transforming them into a heat map (B) and also expressed as a bar graph (C). Data are mean±S.D. (n=4/group). *p<0.05 compared with T/HS, ** p<0.05 compared with T/HS, #p<0.05 compared with T/HS-V, ##p<0.01 compared with T/HS-V.

FIGS. 7A-7D: CD39, CD73, and A2BR regulation of liver enzymes. Aspartate aminotransferase (AST) (A, C) and alanine aminotransferase (ALT) (B, D) levels were determined from plasma spectrophotometrically. Data are mean±S.D. (n=4/group). * p<0.05 compared with T/HS, ** p<0.05 compared with T/HS, #p<0.05 compared with corresponding T/HS-V, ##p<0.01 compared with corresponding T/HS.

FIG. 8: Determination of intestinal injury after T/HS in IEC-specific A2BR deficient mice. Results with IEC-specific VillinCre-A2BRfl/fl mice and control are shown. Data are mean±S.D. (n=4/group). ** p<0.05 compared with T/HS-VillinCre-A2BAR+/+.

### DETAILED DESCRIPTION

A method of treating hemorrhagic shock or hypovolemic shock in a subject, of reducing the likelihood of multi-organ failure in a subject who has had a hemorrhagic shock or hypovolemic shock, or for ameliorating ischemia-reperfusion injury in an organ of a subject who has had a hemorrhagic shock or hypovolemic shock, the method comprising administering to the subject an amount of a CD39 mimic and/or CD73 mimic effective to treat hemorrhagic shock or hypovolemic shock.

In embodiments, the method is for treating hemorrhagic shock or hypovolemic shock in a subject. In embodiments, the method is for reducing the chance of multi-organ failure in a subject who has had a hemorrhagic shock or hypovolemic shock. In embodiments, the method is for ameliorating ischemia-reperfusion injury in an organ of a subject who has had a hemorrhagic shock or hypovolemic shock.

A method for treating or reducing development of acute respiratory distress syndrome in a subject, comprising administering to the subject an amount of a CD39 mimic and/or CD73 mimic effective to treat or reduce development of acute respiratory distress syndrome.

A method comprising:
diagnosing a subject as having hemorrhagic shock or hypovolemic shock, and optionally
diagnosing the patient as not having an infection or as not having a non-sterile shock;
administering to the subject so-diagnosed an amount of a CD39 mimic and/or CD73 mimic effective to reduce or treat an effect of hemorrhagic shock or hypovolemic shock.

In embodiments of the methods, the subject has had a physical trauma-induced hemorrhagic shock.

In embodiments, the subject has a sterile shock or does not have sepsis, septic shock, or shock associated with an infection.

In embodiments, the amount of a CD39 mimic and/or CD73 mimic are administered as a composition comprising the amount of CD39 mimic and/or CD73 mimic and blood or a blood product.

In embodiments, wherein the method is for ameliorating ischemia-reperfusion injury in an organ of a subject who has had a hemorrhagic shock or hypovolemic shock, the organ is a lung, gastrointestinal tract, heart, liver or kidney.

In embodiments, the amount of CD39 mimic and/or CD73 mimic is administered systemically and/or by transfusion. In embodiments, the CD39 mimic and/or CD73 mimic is/are soluble.

In embodiments, no adenosine receptor ligand(s) are administered to the subject.

A method of treating hypovolemic shock in a subject comprising transfusing into the subject having hypovolemic shock an amount of a blood product or blood replacement product which comprises an amount of a CD39 mimic and/or CD73 mimic, sufficient to treat hypovolemic shock.

In embodiments, the blood product or blood replacement product comprises packed red blood cells, whole blood, plasma or platelets.

In embodiments, the methods further comprise identifying the subject as requiring a blood transfusion prior to administration of the CD39 mimic and/or CD73 mimic, and administering the CD39 mimic and/or CD73 mimic in the form of a blood transfusion or blood product transfusion to the subject so-identified.

A composition for treating hemorrhagic shock or hypovolemic shock, or for ameliorating ischemia-reperfusion injury in an organ, comprising:
a blood product or blood replacement product; and
an amount of a CD39 mimic and/or CD73 mimic.

In embodiments, the CD39 mimic is recombinant human apyrase. In embodiments, the CD39 mimic is recombinant human CD39. In embodiments, the CD73 mimic is recombinant human CD73. In embodiments, the composition comprises an amount of both a CD39 mimic and CD73 mimic. In embodiments, the blood or blood product comprises packed red blood cells, whole blood, plasma or platelets.

A method of decreasing liver injury, or reducing an increase in lung permeability and neutrophil infiltration, associated with hypovolemic shock in a subject comprising transfusing into the subject having hypovolemic shock an amount of a CD39 mimic and/or CD73 mimic, sufficient to decrease liver injury, or sufficient to decrease an increase in lung permeability and neutrophil infiltration, associated with hypovolemic shock.

In embodiments, a blood product or blood replacement product which comprises an amount of a CD39 mimic and/or CD73 mimic is administered. In embodiments, liver injury is decreased. In embodiments, an increase in lung permeability and neutrophil infiltration is reduced.

Hemorrhagic shock is well-known in the art, and is a form of hypovolemic shock in which severe traumatic blood loss leads to inadequate oxygen delivery to tissues.

In some embodiments, a CD39 mimic is a molecule or molecules having ectonucleotidase activity on ATP (an ectonucleoside triphosphate diphosphohydrolase-1), but excludes native CD39 in the subject. Examples of CD39 mimics include apyrases from other species other than humans, and recombinant (i.e., synthetically produced) human CD39. Examples of CD39 mimics include non-human apyrase. In some embodiments, a CD73 mimic is a molecule or molecules having ectonucleotidase activity on AMP (as an ecto-5?-nucleotidase), but excludes native CD73 in the subject. Examples of CD73 mimics include recombinant (i.e., synthetically produced) human or non-human CD73. GENBANK Accession Nos. NP-001767, NP-001091645, NP-001157650, NP-_001157651, NP-001157653, and NP-_001157655, which disclose isoforms 1-6), human NTPDase3 (GENBANK Accession NO. NP-_001239), a non-human apyrase, or an enhanced apyrase with improved therapeutic properties such as longer half-life, higher stability, or higher solubility, or higher purity (see U.S, Patent Application Publication No. 2013/0142775, the content of which is incorporated herein by reference), are all examples of molecules having ectonucleotidase activity. GENBANK Accession Nos. NP_001191742, NP_002517, NP_035981, a non-human ecto-5?-nucleotidase, or an enhanced ecto-5?-nucleotidase with improved therapeutic properties such as longer half-life, higher stability, or higher solubility, or higher purity, are all examples of molecules having ecto-5?-nucleotidase activity. In some embodiments, the CD39 mimic is a polypeptide. In some embodiments, the CD73 mimic is a polypeptide. In some embodiments, the CD39 mimic is a polypeptide having four apyrase-conserved regions. Apyrase-conserved regions have been described, for example, in Handa and Guidotti, “Purification and cloning of a soluble ATP-diphosphohydrolase (apyrase) from potato tubers (Solanum tuberosum)” Biochem Biophys Res Commun, 1996; 218 (3): 916-23 (i.e., Reference #45), the content of which is incorporated herein by reference. In some embodiments, the CD39 mimic is soluble and not membrane-bound. In some embodiments, the CD73 mimic is soluble and not membrane-bound.

The invention provides for CD39 mimic and/or CD73 mimic polypeptides which may be purified or recombinant. The terms “substantially purified polypeptide” or “purified polypeptide” refer to a polypeptide that has generally been separated from the lipids, nucleic acids, other peptides, and other contaminating molecules with which it is associated in a cell in which it is produced or in its native state. Preferably, the substantially purified polypeptide is at least 60% free, more preferably at least 75% free, and more preferably at least 90% free from other components in the cell in which it is produced or with which it is naturally associated.

The term “recombinant” in the context of a polypeptide refers to the polypeptide when produced by a cell, or in a cell-free expression system, in an altered amount or at an altered rate, compared to its native state if it is produced naturally. In one embodiment the cell is a cell that does not naturally produce the polypeptide. However, the cell may be a cell which comprises a non-endogenous gene that causes an altered amount of the polypeptide to be produced. A recombinant polypeptide of the invention includes polypeptides in the cell, tissue, organ or organism, or cell-free expression system, in which it is produced i.e., a polypeptide which has not been purified or separated from other components of the transgenic (recombinant) cell in which it was produced, and polypeptides produced in such cells or cell-free systems which are subsequently purified away from at least some other components.

The terms “polypeptide” and “protein” are generally used interchangeably.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence may be determined by GAP analysis (Needleman and Wunsch, 1970; GCG program) with, for example, parameters of a gap creation penalty=5, and a gap extension penalty=0.3. In some embodiments, the GAP analysis aligns two sequences over their entire length.

Preferably, the polypeptide has an enzymatic activity of at least 10% of the activity of the reference polypeptide.

As used herein a “biologically active” fragment is a portion of a polypeptide of the invention which maintains a defined activity of a full-length reference polypeptide, for example possessing ectonucleotidase, apyrase, 5′-nucleotidase, ecto-5′-nucleotidase, ATP-diphosphatase, adenosine diphosphatase, ADPase, and/or ATP diphosphohydrolase activity or other enzyme activity. Biologically active fragments as used herein exclude the full-length polypeptide. Biologically active fragments can be any size portion as long as they maintain the defined activity. Preferably, the biologically active fragment maintains at least 10% of the activity of the full length protein.

In some embodiments, the CD39 mimic or CD73 mimic is a polypeptide/enzyme comprising an amino acid sequence which has at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a known CD39 or CD73 enzyme, respectively.

In some embodiments, the CD39 mimic is a polypeptide/enzyme comprising an amino acid sequence which has at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to an enzymatic activity portion of a CD39 enzyme, preferably a portion which confers apyrase activity or ectonucleotidase activity, preferably on ATP.

In some embodiments, the CD73 mimic is a polypeptide/enzyme comprising an amino acid sequence which has at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to an enzymatic activity portion of a CD73 enzyme, preferably a portion which confers ectonucleotidase activity, preferably on AMP.

In some embodiments the CD39 mimic and/or CD73 mimic comprises one or more amino acid portions in addition to an ectonucleotidase activity portion, e.g., a signal peptide, an affinity tag, a cleavage peptide, an amino acid linker, an additional domain, etc.

In an embodiment, the substantially purified and/or recombinant CD39 mimic of the invention differs in amino acid sequence from a wild-type human CD39 enzyme.

In another embodiment, the substantially purified and/or recombinant CD73 mimic of the invention differs in amino acid sequence from a wild-type human CD73 enzyme.

Amino acid sequence mutants of the polypeptides of the defined herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. For example, amino acids of a known CD39 or CD73 enzyme, or portion thereof (e.g., an apyrase or ectonucleotidase enzymatic portion), may be altered by amino acid deletion, insertion, and/or substitution to generate a CD39 mimic or CD73 mimic, respectively.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they possess apyrase and/or ectonucleotidase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Substitution mutants have at least one amino acid residue in the polypeptide molecule removed and a different residue inserted in its place. In some embodiments, amino acids are substituted in a relatively conservative manner. Details of conservative amino acid changes are provided in Table 1.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Also included within the scope of the invention are polypeptides defined herein which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides can be produced in a variety of ways, including production and recovery of natural polypeptides, production and recovery of recombinant polypeptides, and chemical synthesis of the polypeptides. In one embodiment, a recombinant polypeptide is produced by culturing a cell capable of expressing the polypeptide under conditions effective to produce the polypeptide. The recombinant polypeptide may subsequently be secreted from the cell and recovered, or extracted from the cell and recovered, and is preferably purified away from contaminating molecules. It may or may not be further modified chemically or enzymatically. A preferred cell to culture is a recombinant cell defined herein. Effective culture conditions include, but are not limited to, effective media, bioreactor, temperature, pH and oxygen conditions that permit polypeptide production. An effective medium refers to any medium in which a cell is cultured to produce a polypeptide defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

The CD39 mimic and/or CD73 mimic as a therapeutic agent disclosed herein can be lyophilized and/or freeze dried and are reconstituted for use. Compositions or pharmaceutical compositions comprising the CD39 mimic and/or CD73 mimic disclosed herein can comprise stabilizers to prevent loss of activity or structural integrity of the enzyme due to the effects of denaturation, oxidation or aggregation over a period of time during storage and transportation prior to use. Where a composition or pharmaceutical composition of the present disclosure is used as an injection, infusion, or transfusion, it is desirable to have a pH value in an approximately neutral pH range or human blood pH range, it is also advantageous to minimize surfactant levels to avoid bubbles in the formulation which are detrimental for injection into subjects. In an embodiment, the composition or pharmaceutical composition is suitable for intravenous, intramuscular, intraperitoneal, intradermal, intraorgan, and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

When not being used in a blood or blood product but as a standalone pharmaceutical composition, examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the compositions can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy) ethoxy]ethyl (E)-octadec-9-enoate), disodium edetate dehydrate, sucrose, monobasic sodium phosphate monohydrate, and dibasic sodium phosphate dihydrate.

In embodiments, administration can be via transfusion. In embodiments, administration can be systemic. In embodiments, administration can be local or direct intraorgan. In embodiments, administration can be intramuscular or subcutaneous. Administration can be oral or parenteral. In embodiments, administration is via infusion or injection. In embodiments, administration is intrathecal or intraventricular. In embodiments, administration is intravenous.

In embodiments where the subject is identified as being in need of a blood transfusion, a blood transfusion is an instance in which a patient requires at least one unit of packed red blood cells (pRBC). One unit of pRBC has a volume of approximately 450 ml. pRBC are red blood cells that have been collected, processed, and stored in bags as blood product units available for blood transfusion purposes. The red blood cells are mixed with an anticoagulant and storage solution which provides nutrients and aims to preserve the viability and functionality of the cells, which are stored at refrigerated temperatures. In embodiments, blood as used herein means human blood or blood obtained from a human. In embodiments, blood products as used herein can include human blood-based products or artificial blood replacement-based products.

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

Definitions: The terms used in this specification generally have their ordinary meanings in the art, within the context of this invention and the specific context where each term is used. Certain terms are discussed herein to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means a mammal. Mammals include canines, felines, rodents, bovine, equines, porcines, ovines, and primates including humans. Thus, in embodiments the compositions and/or methods can be used in human medicine or also in veterinary medicine, e.g., to treat companion animals, farm animals, laboratory animals in zoological parks, and animals in the wild. In embodiments the compositions and/or methods are particularly desirable for human medical applications, especially in trauma situation. In a preferred embodiment the subject is a human.

The terms “treat”, “treatment” of a disease or condition, and the like refer to slowing down, relieving, ameliorating or alleviating at least one of the symptoms of the condition.

The terms “therapeutically effective amount” or “amount effective to” encompasses, unless otherwise indicated, an amount sufficient to ameliorate or inhibit a symptom or sign of the medical condition. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Trauma and a subsequent hemorrhagic shock (T/HS) result in insufficient oxygen delivery to tissues and multiple organ failure. Extracellular adenosine, which is a product of the extracellular degradation of adenosine 5′ triphosphate (ATP) by the membrane-embedded enzymes CD39 and CD73, is organ protective, as it participates in signaling pathways which promote cell survival and suppress inflammation through adenosine receptors including the A2BR.

In this study, we investigated the role of CD39, CD73, and the A2BR in regulating T/HS-induced organ failure. T/HS upregulated the expression of CD39, CD73, and the A2BR in organs. ATP and adenosine levels increased after T/HS in bronchoalveolar lavage fluid. CD39, CD73, and A2BR mimics/agonists alleviated lung and liver injury. Antagonists or the absence of CD39, CD73, and A2BR exacerbated lung injury, inflammatory cytokines, and chemokines as well as macrophage and neutrophil infiltration and accumulation in the lung. Agonists reduced the levels of the liver enzymes aspartate transferase and alanine transaminase in the blood, whereas antagonist administration or genetic absence of CD39, CD73, and A2BR enhanced enzyme levels. In addition, intestinal epithelial cell-specific deficient VillinCre-A2BRfl/fl mice showed increased intestinal injury compared to their wild-type VillinCre controls. In conclusion, the CD39-CD73-A2BR axis protects against T/HS-induced multiple organ failure.

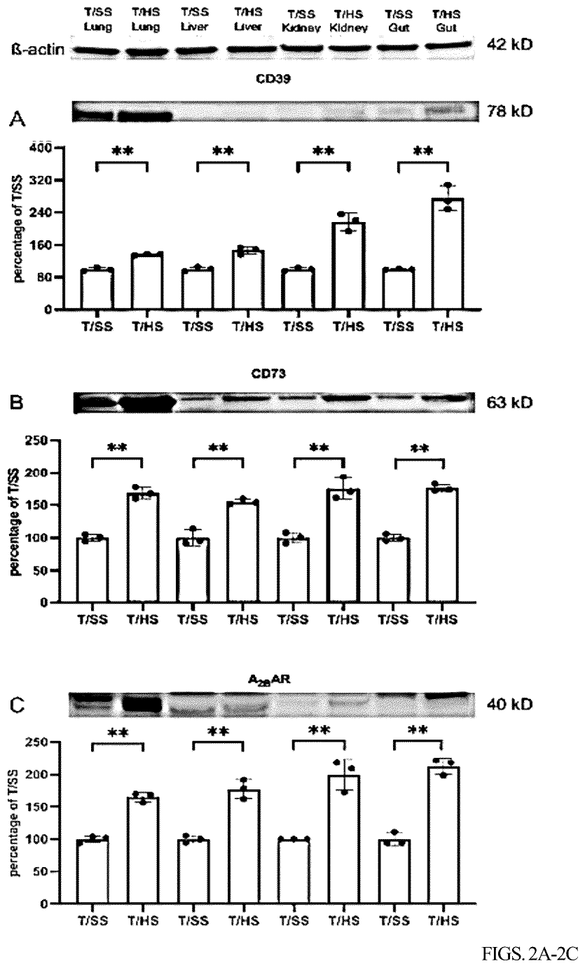

T/HS increases CD39, CD73, and A2BR expression in lung, liver, kidney, and gut: T/HS increased the expression of CD39, CD73, and the A2BR in the lung, liver, kidney, and gut when compared to T/SS (FIGS. 2A-2C).

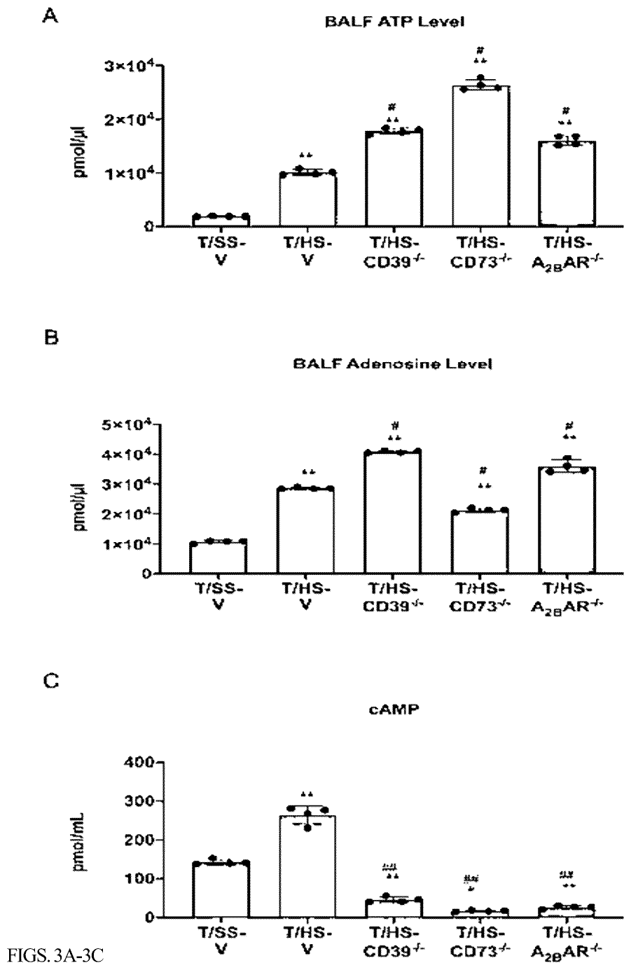

Role of CD39, CD73, and A2BR in regulating ATP, adenosine, and cAMP levels after T/HS: We found that T/HS increased ATP, adenosine, and cAMP levels in BALF samples (FIG. 3, A-C). The absence of CD39, CD73, and A2BR further increased T/HS-induced ATP and adenosine (FIGS. 3, A and B). T/HS increased cAMP compared to T/SS and cAMP levels decreased in the absence of CD39, CD73, and A2BR in T/HS BALF (FIG. 3C).

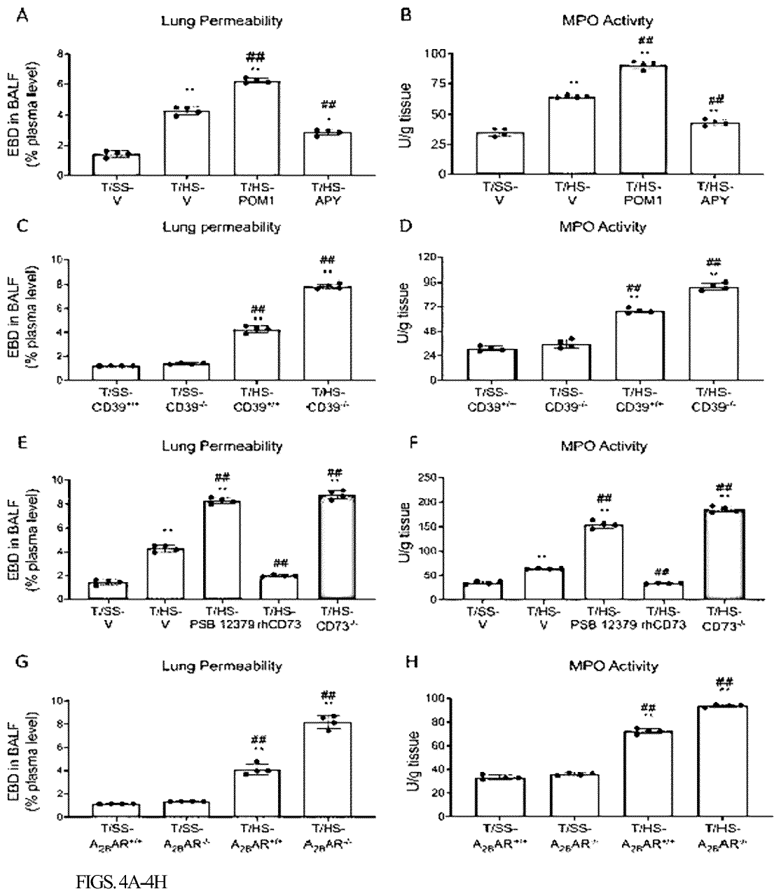

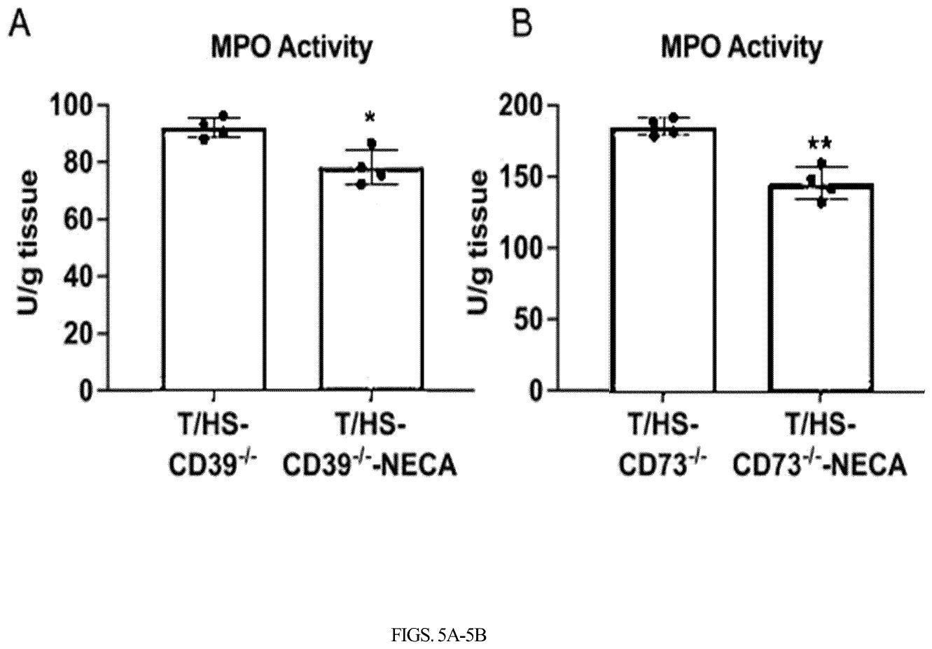

CD39, CD73, and the A2BAR suppress lung permeability and neutrophil infiltration: Treatment with POM1 exacerbated and treatment with apyrase moderated T/HS-induced lung injury and neutrophil sequestration after T/HS (FIGS. 4, A and B). Genetic deficiency of CD39, CD73, and the A2BR increased lung permeability and neutrophil sequestration (FIG. 4, C—H). PSB 12379 increased lung injury and neutrophil infiltration while rhCD73 decreased these parameters (FIGS. 4E and 4F). We then examined whether adenosine receptor stimulation was able to rescue the increased injury in CD39^{-/-} and CD73^{-/-} mice. NECA pretreatment decreased neutrophil infiltration in CD39^{-/-} and CD73^{-/-} mice after T/HS (FIGS. 5A and 5B). The lung injury score increased in T/HS vs. T/SS mice and was further increased in the absence of CD39, CD73, and A2BR.

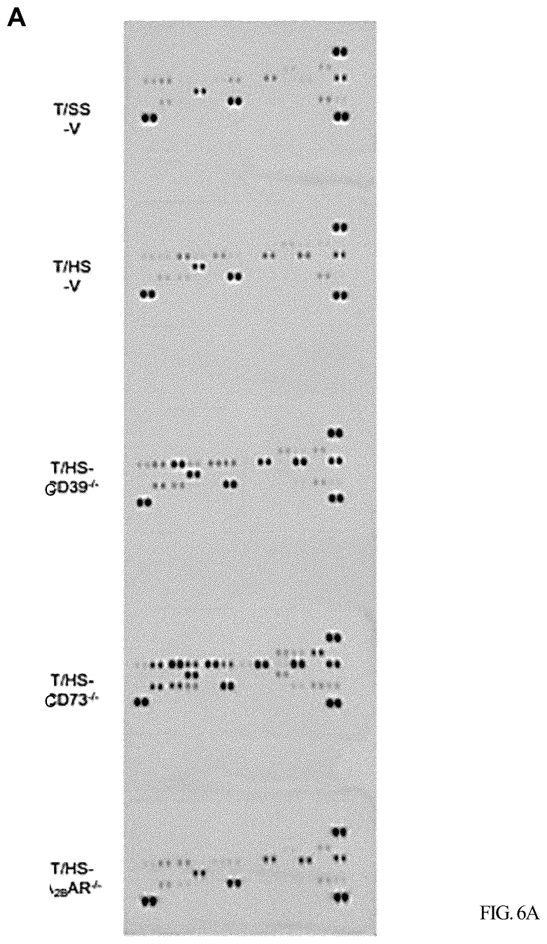

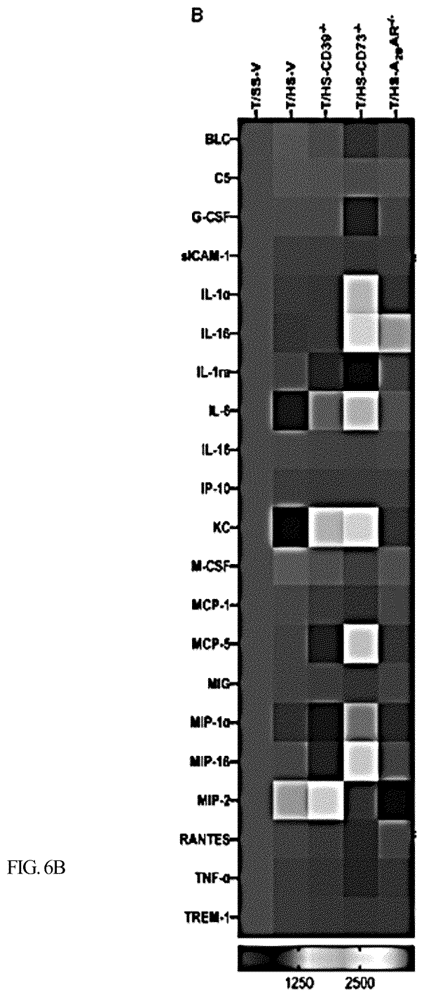

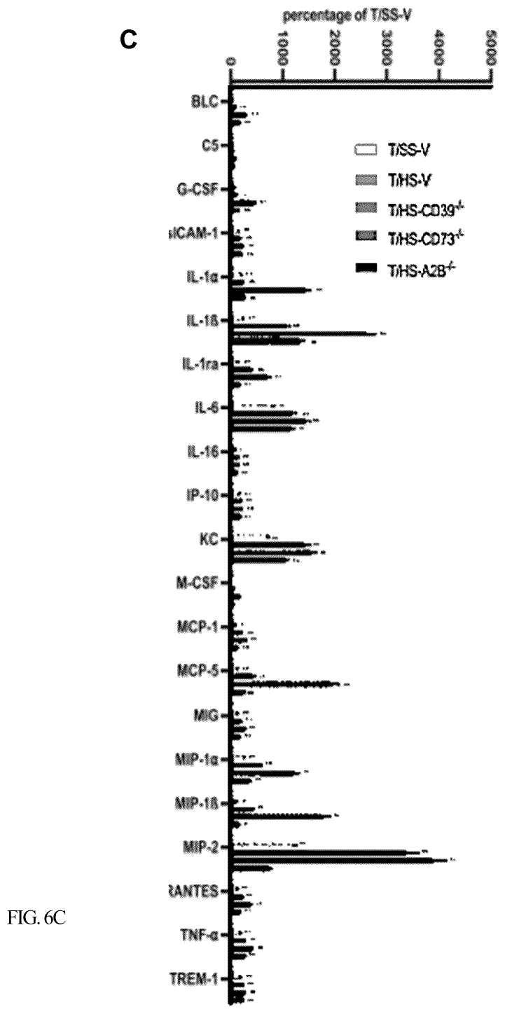

Regulation of lung cytokine levels by CD39, CD73, and the A2BR: Using an antibody array, we assessed the relative expression levels of 40 different cytokines and chemokines including CXCL13/BLC/BCA-1, C5a, G-CSF, GM-CSF, CCL1/1-309, CCL11/Eotaxin, ICAM-1, IFN-gamma, IL-1 alpha/IL-1F1, IL-1 beta/IL-1F2, IL-1ra/IL-1F3, IL-2, IL-3, IL-4, IL-5, IL-6, IL-7, IL-10, IL-12 p70, IL-13, IL-16, IL-17, IL-23, IL-27, CXCL10/IP-10, CXCL11/I-TAC, CXCL1/KC, M-CSF, CCL2/JE/MCP-1, CCL12/MCP-5, CXCL9/MIG, CCL3/MIP-1 alpha, CCL4/MIP-1 beta, CXCL2/MIP-2, CCL5/RANTES, CXCL12/SDF-1, CCL17/TARC, TIMP-1, TNF-alpha, and TREM-1 in the lung (FIG. 6A). We found that levels of pro-inflammatory cytokines and chemokines (BLC, C5, G-CSF, SICAM-1, IL-1α, IL-1ß, IL-1ra, IL-6, IL-16, IP-10, KC, M-CSF, MCP-1, MCP-5, MIG, MIP-1α, MIP-1ß, MIP-2, RANTES, TNF-α and TREM-1) increased in the absence of CD39, CD73, and A_{2B}Rs in the lung after T/HS (FIGS. 6B and 6C).

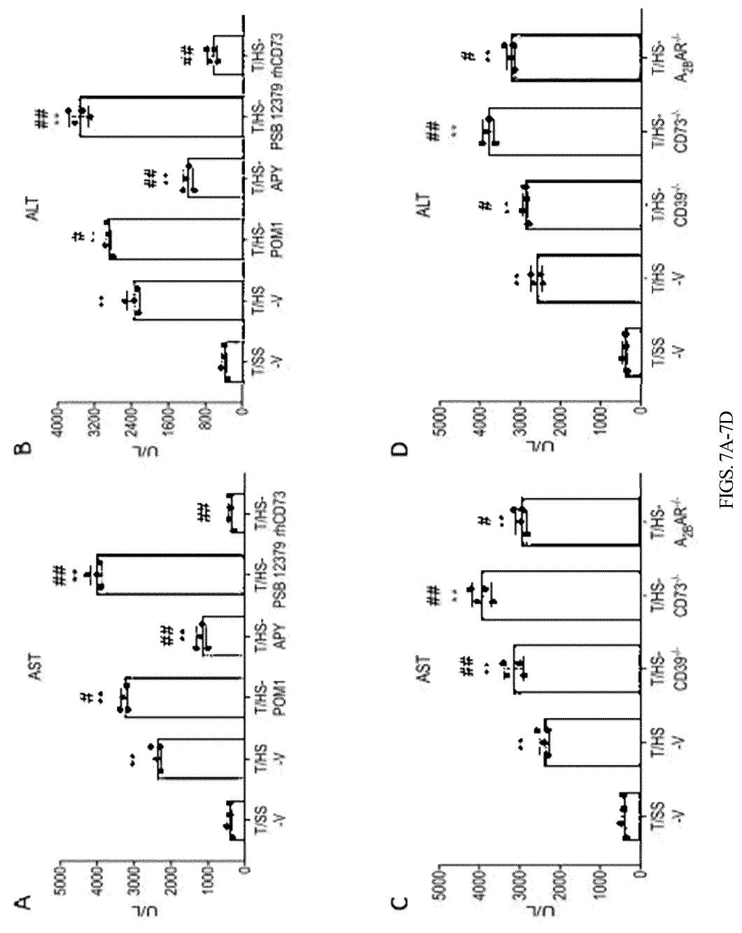

Role of CD39, CD73 and A2BR in regulating T/HS-induced liver injury: Apyrase or rhCD73 treatment suppressed T/HS-enhanced AST and ALT levels, indicating decreased liver injury, while POM1 and PSB 12379 increased AST and ALT (FIGS. 7, A and B). In addition, the absence of CD39, CD73, and A_{2B}Rs led to elevated AST and ALT levels (FIGS. 7, C and D).

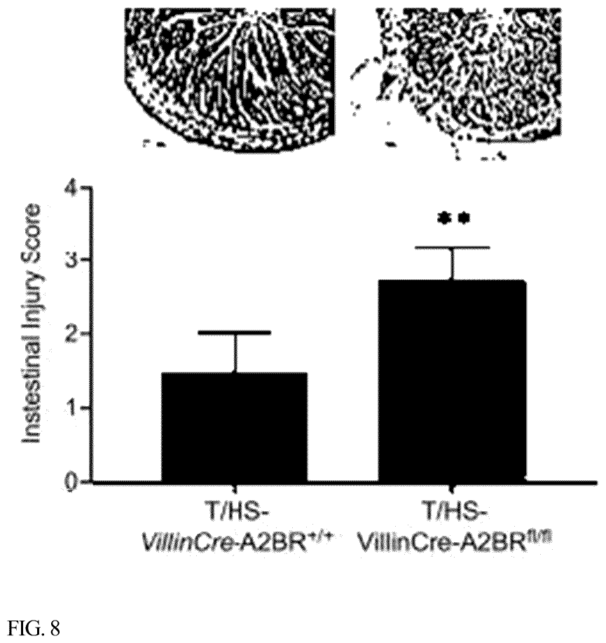

Regulation of intestinal injury by CD39, CD73, and the A_{2B}R: T/HS increased gut injury compared to T/SS. Global CD39, CD73, and A_{2B}R deficiency failed to influence the extent of gut injury in T/HS mice. However, Villin^{Cre}-A_{2B}AR^{fl/fl} mice had increased gut injury when compared to their WT control Villin^{Cre}-A_{2B}AR^{+/+} mice after T/HS. (FIG. 8).

Role of CD39, CD73 and A_{2B}Rs in regulating plasma IL-6 and IL-10 levels: As compared to vehicle-treated T/HS mice, POM1-treated mice had higher plasma IL-6 levels and similar plasma IL-10 levels. Also, apyrase-treated T/HS mice had lower plasma IL-6 levels and increased plasma IL-10 levels compared to vehicle-treated T/HS mice. CD39-mice after T/HS had increased IL-6 and decreased IL-10 compared to wild-type controls.

When compared to vehicle-treated T/HS mice, PSB 12379-treated T/HS animals had increased plasma IL-6 and reduced IL-10 levels and rhCD73-treated T/HS mice had decreased plasma IL-6 and increased plasma IL-10. Plasma IL-6 was increased and IL-10 decreased in CD73^{-/-} compared to wild-type T/HS mice. A_{2B}AR^{-/-} mice exhibited higher plasma IL-6 levels while having lower plasma IL-10 levels compared to the A_{2B}R group subjected to T/HS.

CD39, CD73, and A_{2B}Rs moderate macrophage accumulation in the lung: CD39, CD73, and A_{2B}R deficiency all increased macrophage accumulation following T/HS, as indicated by immunohistochemistry.

Effect of the absence of CD39, CD73 and A_{2B}Rs on the expression of survival-related proteins: To investigate the role of CD39, CD73, and the A_{2B}R in regulating the expression of survival-related proteins, we analyzed p-PTEN and MMP-9 expression after T/HS. The absence of CD39, CD73, and A_{2B}Rs all decreased p-PTEN and increased MMP-9 expression levels in lung tissue.

Discussion: We demonstrate that exogenous stimulation of the A_{2A}R decreases organ injury after T/HS. In addition, we found that inhibition or the genetic absence of A_{2A}Rs leads to increased lung permeability, MPO level, and augmented liver enzymes (32). Our current studies demonstrate for the first time that ATP is released into the extracellular space during T/HS. In agreement with previous studies implicating extracellular ATP as the primary source for adenosine generation in ischemia of particular organs (4, 7-9), our studies targeting CD39 and CD73 demonstrate that ATP is also the likely source of extracellular adenosine following T/HS, a condition whose pathophysiology is much more complex than that of ischemia of the various organs. In addition, we demonstrate for the first time that the A_{2B}R activated by endogenous adenosine protects organs from T/HS-induced organ injury.

CD39 is a transmembrane protein found in the spleen, thymus, lung, and placenta and is largely linked with immune cell populations and endothelial cells (10, 33, 34). Several proinflammatory cytokines, oxidative stress, and hypoxia affect the CD39 expression (35). Studies indicate that CD39 is organ protective against ischemia/reperfusion injury, sepsis, and heart disease (36-43). For example, in CD39 deficient mice the organ injury and inflammation that followed cardiac (40), renal (37), hepatic (16, 38), and intestinal (44) ischemia-reperfusion injury were more severe than in the corresponding wild-type mice. A common thread in these studies was that exogenous supplementation with potato apyrase reversed the increased ischemic organ injury of CD39 deficient mice. Potato apyrase is widely used as a CD39 mimic, because it has an amino acid sequence that is highly homologous to CD39, particularly within its four apyrase-conserved regions (45). Given that T/HS causes global ischemia-reperfusion injury, our results demonstrating protective effects for CD39 in T/HS correspond well with earlier results with ischemia-reperfusion injury of the various organs. Given the commonalities of mechanisms for the injury of the various organs, it will be of interest to determine whether protection in the various models can be traced back to one particular cell type expressing CD39 or whether protection depends on different cell types based on the organ.

CD73 regulates multiple events such as cellular hemostasis and tissue injury and is found in a variety of tissues, including the colon, brain, kidney, liver, lung, and heart (46); on leukocytes derived from peripheral blood, spleen, lymph nodes, thymus, and bone marrow (46); as well as on endothelium (47). The expression and function of this enzyme are upregulated under hypoxic conditions (48, 49), and by several proinflammatory mediators, such as transforming growth factor (TGF)-β, interferons (IFNs), TNF-α, IL-1β, and prostaglandin E₂ (50, 51). Similar to CD39, CD73 is also organ protective against hypoxic and ischemic conditions (46, 49, 52). The increased vascular permeability and polymorphonuclear neutrophil extravasation noted in hypoxic CD73 deficient mice were reversed by stimulating ARs by exogenous administration of 5′-(N-ethylcarboxamido) adenosine (NECA), a general AR agonist, or by exogenous reconstitution with a soluble CD73-like nucleotidase (46). In this respect, our data with the NECA-mediated “rescue” of the increased neutrophil infiltration of lungs observed in CD73 (and CD39) deficient mice indicate that CD73 together with CD39 deliver adenosine to adenosine receptors to dampen inflammation and injury in T/HS.

The A_{2B}R activates intracellular signaling mechanisms such as cAMP (53). Elevated intracellular levels of cAMP are frequently linked to anti-inflammatory outcomes, including the synthesis of IL-10, the inhibition of leukocyte infiltration, and pro-inflammatory cytokines (54). We have previously shown that A_{2B}R-deficient mice have higher mortality and increased pro-inflammatory cytokines such as IL-6, TNF-α and MIP-2 in sepsis (55). Also, posttreatment with A_{2B}R agonist BAY 60-6583 decreases lung permeability but not neutrophil infiltration into the lung in a rat model of T/HS (6).

### Materials and Methods

### Ethical Statements and Animals:

Adult (8-12-week-old) CD39−/−, A2B R−/−, VillinCre-A2BARfl/fl, and their WT control VillinCre-A2BAR+/+ mice were bred at and wild-type C57BL/6J mice obtained from Charles River (Wilmington, MA, USA). CD73^{-/-} (B6.129S1-Nt5e^{tm1Lft}/J) mice were purchased from Jackson Laboratory (Bar Harbor, ME, USA). CD73^{-/-} mice were bred and maintained in a specific pathogen-free Columbia University animal facility until being used in experiments. Mice had access to food and water ad libitum. They were kept in a room with a 12-h light-dark cycle under nonspecific pathogen-free conditions.

### Drugs:

The selective CD39 inhibitor sodium polyoxotungstate (POM1), CD73 inhibitor PSB 12379 (N6-Benzyl-α,β-methyleneadenosine 5′-diphosphate disodium salt), and adenosine receptor agonist 1-(6-Amino-9H-purin-9-yl)-1-deoxy-N-ethyl-β-D-ribofuranuronamide (NECA) were from Tocris (Bristol, UK). The CD39 mimic potato apyrase was from Sigma, and recombinant human (rh) CD73 was from Daresbury Proteins (Warrington, UK).

### Study Design and Induction of Trauma Hemorrhagic Shock:

In one set of studies, wild-type mice were randomly assigned into the following groups: trauma/sham shock (T/SS) receiving vehicle (saline), trauma/hemorrhagic shock (T/HS) receiving vehicle (saline), T/HS receiving POM1 (5 mg/kg), T/HS receiving apyrase (125 U/kg), T/HS receiving PSB 12379 (50 mg/kg), and T/HS receiving rhCD73 (2 mg/kg). In addition, in another group of studies, CD39^{-/-}, CD73^{-/-}, A_{2B}AR^{-/-} and wild-type type mice were subjected to T/SS or T/HS. Some CD39^{-/-} and CD73^{-/-} were pretreated with NECA (0.0025 mg/kg). In pharmacological experiments, mice were pretreated intraperitoneally (30 min before T/SS or T/HS) with various agents.

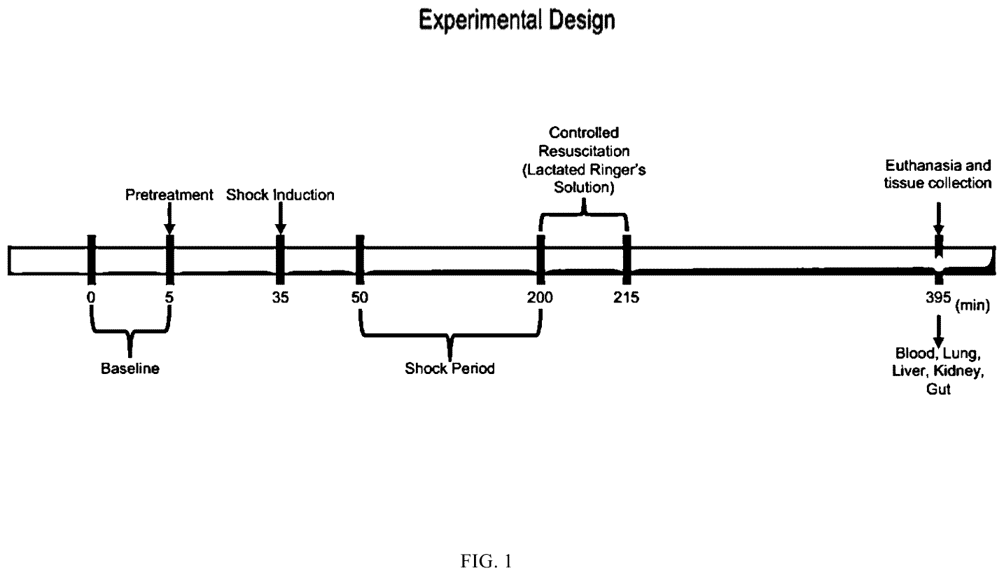

The mice were given anesthesia using 1% isoflurane and their rectal temperature was maintained between 36.5 and 37.5° C. using a feedback-controlled homeothermic blanket heating system (Sumno-suite, Kent Scientific). Hemorrhagic shock was induced using a fixed-pressure model. Initially, a midline laparotomy of 2 cm was performed on the anesthetized mice, which was later closed with a 4-0 silk suture (034902, Covetrus, USA). Following this, catheters were placed in the right and left femoral arteries for monitoring blood pressure and blood withdrawal, respectively. A sterile 1-ml syringe with a 30G needle attached to PE-10 tubing filled with 0.2 ml of 1% heparinized saline was used for blood withdrawal, and each mouse received 1U heparin. Blood pressure was monitored using a continuous blood-pressure monitoring system (Powerlab 8/30, ADInstruments, Colorado Springs, CO, USA). After 5 minutes of baseline blood pressure recording, a drug or vehicle was administered to the mice, followed by inducing shock for a period of 2.5 hours, during which the blood pressure was maintained between 28-32 mmHg by withdrawing or reinfusing the shed blood. At the end of the shock period, the mice were resuscitated with Ringer's Lactate at three times the amount of shed blood for 15 minutes (32). Three hours after resuscitation, the mice were euthanized and bronchoalveolar lavage fluid (BALF), blood, and tissue samples were collected. T/SS animals were exposed to the same procedures as other animals except for blood withdrawal (FIG. 1).

### Tissue Preparation for Evaluating Neutrophil Sequestration, Mouse Cytokine Array, and Western Blot Analysis:

Lung, liver, kidney, and gut samples were pooled from 4 mice in the same group, homogenized, sonicated, and treated with a 1×RIPA Buffer with Protease/phosphatase inhibitor cocktail (P8340, Sigma, USA), and the resulting homogenate was centrifuged at 13.000× g for 10 minutes at 4° C. Total protein content was determined by using Qubit 4.0 Fluorometer (Thermo Fisher, USA) as per the manufacturer's instructions.

### Collection of BALF Samples and Determination of BALF ATP, Adenosine, and Camp Levels:

Three hours after the end of resuscitation the mice were euthanized, and the trachea was isolated for collecting BALF samples. Briefly, after a small incision a syringe with a 23G needle filled with 1 ml of sterile saline was inserted into the trachea. After centrifugation (1500×g for 15 min at 4° C.) the supernatant was used for the assays (32). ATP (ab83355, Abcam, USA) and cAMP (KGE012B, R&D Systems, USA) in BALF were measured by a colorimetric assay. Adenosine was measured by a fluorometric assay (ab211094, Abcam, USA) according to the manufacturer's instructions.

### Lung Permeability Measurements:

Mice were re-anesthetized with isoflurane 3 hours after the end of resuscitation. Evans blue dye (EBD) technique was used to determine lung permeability. EBD was administered through the tail vein, and about 1 ml of blood was withdrawn from the tail artery five minutes later. The supernatant of BALF was assayed at 620 nm spectrophotometrically. The amount of Evans blue dye in the BALF was then expressed as a proportion of the amount in the plasma. (5, 32).

### Determination of Pulmonary Neutrophil Sequestration:

To evaluate neutrophil sequestration in the lung following T/SS or T/HS, myeloperoxidase (MPO) activity was assessed using an MPO activity kit (MAK068, Sigma, USA). The supernatant of lung lysates was assayed according to the manufacturer's instructions (32).

### Mouse Cytokine Array:

To examine cytokine and chemokine expression in lung samples, a total of 200 μg protein was analyzed using the Proteome Profiler Mouse Cytokine Array Panel A Kit (ARY006, R&D Systems, USA). The expression of cytokines and chemokines was determined densitometrically using Fiji software after subtracting the negative control's average signal. Values were expressed as % of T/SS (56).

### Measurement of AST and ALT Levels:

Plasma samples were analyzed to determine the levels of aspartate aminotransferase (AST) and alanine aminotransferase (ALT). To do this, the samples were diluted with AST (TR70121 Thermo Fisher, USA) and ALT (TR71122 Thermo Fisher, USA) reagents at a 1:10 ratio, and the resulting light signal was measured using a spectrophotometer at 340 and 405 nm (32).

### Determination of IL-10 and IL-6 Levels by Enzyme-Linked Immunosorbent Assay

IL-6 and IL-10 levels were measured in plasma samples using enzyme-linked immunosorbent assay (ELISA) kits (DY406, DY417; R&D Systems Minneapolis, USA) according to the manufacturer's instructions (32).

### Histopathological Assessment of Lung and Intestinal Injury:

Sections of lung and gut were cut at 5 μm thickness, stained with the hematoxylin-eosin (H&E) method, and scanned with a Lecia AT2 slide scanner (Leica, USA). Lung sections were evaluated histopathologically in terms of the parameters: 1: neutrophils in the alveolar spaces, 2: neutrophils in the interstitial space, 3: hyaline membranes, 4: proteinaceous debris filling in the airspaces, 5: alveolar septal thickening by a blind observer (57). Ileum samples were evaluated histopathologically in terms of the parameters: 1: desquamation and necrosis of upper ⅓ villi, 2: progressive peel off mid of villi, 3: peel of lower ⅓ villi and necrosis of crypt cells, 4: necrosis of ⅔ crypt cells, 5: complete loss of basal crypts.

### Western Blot:

CD39, CD73, A_{2B}R, phosphorylated-Phosphatase and Tensin (p-PTEN) and matrix metalloproteinase-9 (MMP-9) protein levels after T/SS or T/HS were determined using western blotting. Thirty micrograms of protein per sample were size-fractioned using 4-20% Mini-Protean TGX Stain-Free (4568093, Bio-Rad, Life Sciences Research) electrophoresis gel and then transferred to a PVDF membrane (1620174, Bio-Rad, Life Sciences Research) using Mini Trans-Blot Electrophoretic Transfer System (1703930, Bio-Rad, Life Sciences Research). The membranes were first blocked with a blocking solution composed of 5% non-fat dry milk in 50 mM Tris-buffered saline containing 0.1% Tween 20 (TBS-T) for 1 hour at room temperature. Following this, the membranes were washed with 50 mM TBS-T and then incubated overnight with a rabbit monoclonal anti-CD39 (ab223842, Abcam, USA), rabbit polyclonal anti-CD73 (ab175396, Abcam, USA), rabbit polyclonal anti-A_{2B}R (ab229671, Abcam, USA), rabbit polyclonal p-PTEN (9551, Cell Signaling, USA) and rabbit polyclonal anti-MMP-9 (ab38898) at a dilution 1:1000, 1:1000, 1:500, 1:1000 and 1:2000 respectively. The following day, the membranes were washed with TBS-T and then incubated with horseradish peroxidase (HRP)-conjugated goat anti-rabbit secondary antibody (ab97051, Abcam, USA) at a dilution of 1:5000 in the blocking solution for 2 hours at room temperature. To ensure the protein loading was consistent, the membranes were stripped and re-probed with an HRP-conjugated anti-β actin antibody (ab20272, Abcam, USA). The membranes were then developed using Clarity Western ECL Substrate Kit (1708280, Bio-Rad, Life Sciences Research) (32).

### Immunofluorescence Staining:

Macrophage staining was performed as previously described (56). Lung sections (5 μm) were fixed in 4% paraformaldehyde in PBS, washed, and immersed for 30 minutes in 0.1 M PBS containing 0.3% Triton-X-100 (PBS-T)/10% normal goat serum. Sequentially 2 lung sections from each sample were incubated overnight at 4° C. with AlexaFlour 488-conjugated monoclonal mouse anti-F4/80 (53-4801-82, Thermo Fisher, USA). The following day, sections were incubated with 4′,6-diamidino-2-phenylindole (DAPI) (D9542, Sigma, USA) at room temperature for 5 minutes. Sections were analyzed using confocal laser scanning microscopy (Zeiss LSM 900, Jena, Germany). Eight different regions of interest (ROI) from the sections were calculated. The percentage of macrophages in the lung tissue was calculated by dividing the results obtained from T/HS by the results of T/SS and then multiplying the quotient by 100.

### Statistical Analysis:

The Shapiro-Wilk test was used to determine the normality of the data in each group, which demonstrated a normal distribution. Statistical analysis was conducted using Graph-Pad Prism (San Diego, CA, USA). One-way ANOVA or t-test was performed followed by Tukey's test as appropriate. The results were presented as mean±S.D. values, and a p-value of less than 0.05 was considered statistically significant.

### Study Approval:

All procedures on mice were performed under Columbia University Institutional Animal Care and Use Committee (IACUC) approval.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### REFERENCES

1. Cannon, J W Hemorrhagic Shock. N Engl J Med, 2018; 378 (19): 1852-1853.
2. Huan, Z, et al. PTPRO knockdown protects against inflammation in hemorrhage shock-induced lung injury involving the NF-kappaB signaling pathway. Respir Res, 2022; 23 (1): 195.
3. Kao, R L, et al. Induction of acute lung inflammation in mice with hemorrhagic shock and resuscitation: role of HMGB1. J Inflamm (Lond), 2014; 11 (1): 30.
4. Valade, G, et al. Therapeutic Potential of Mesenchymal Stromal Cell-Derived Extracellular Vesicles in the Prevention of Organ Injuries Induced by Traumatic Hemorrhagic Shock. Front Immunol, 2021; 12:749659.
5. Hasko, G, et al. Adenosine A2A receptor activation reduces lung injury in trauma/hemorrhagic shock. Crit Care Med, 2006; 34 (4): 1119-25.
6. Koscso, B, et al. Stimulation of A2B adenosine receptors protects against trauma-hemorrhagic shock-induced lung injury. Purinergic Signal, 2013; 9 (3): 427-32.
7. Chen, Y, et al. Inhibition of Neutrophils by Hypertonic Saline Involves Pannexin-1, CD39, CD73, and Other Ectonucleotidases. Shock, 2015; 44 (3): 221-7.
8 Ham, PB, 3rd and R Raju Mitochondrial function in hypoxic ischemic injury and influence of aging. Prog Neurobiol, 2017; 157:92-116.
9. Wang, Y, et al. CD73 or CD39 Deletion Reveals Different Mechanisms of Formation for Spontaneous and Mechanically Stimulated Adenosine and Sex Specific Compensations in ATP Degradation. ACS Chem Neurosci, 2020; 11 (6): 919-928.
10. Antonioli, L, et al. CD39 and CD73 in immunity and inflammation. Trends Mol Med, 2013; 19 (6): 355-67.
11. Di Virgilio, F, et al. Purinergic signaling, DAMPs, and inflammation. Am J Physiol Cell Physiol, 2020; 318 (5): C832-C835.
12. Hasko, G, et al. Adenosine receptors: therapeutic aspects for inflammatory and immune diseases. Nat Rev Drug Discov, 2008; 7 (9): 759-70.
13. Zhang, ×, et al. Hypoxia-inducible factor-1alpha protects the liver against ischemia-reperfusion injury by regulating the A2B adenosine receptor. Bioengineered, 2021; 12(1): 3737-3752.
14. Chen, J F, et al. Adenosine receptors as drug targets-what are the challenges? Nature Reviews Drug Discovery, 2013; 12 (4): 265-286.
15. Reutershan, J, et al. Adenosine and inflammation: CD39 and CD73 are critical mediators in LPS-induced PMN trafficking into the lungs. Faseb Journal, 2009; 23 (2): 473-482.
16. Wang, S, et al. The role of the CD39-CD73-adenosine pathway in liver disease. J Cell Physiol, 2021; 236 (2): 851-862.
17. Junger, W G Immune cell regulation by autocrine purinergic signalling. Nat Rev Immunol, 2011; 11 (3): 201-12.
18. Rai, R, Cross Talk of Purinergic and Immune Signaling: Implication in Inflammatory and Pathogenic Diseases, in Purinergic System, M. D. Bagatini, Editor. 2022, IntechOpen: United Kingdom. p. 1-29.
19. Sun, Y and PB Huang Adenosine A (2B) Receptor: From Cell Biology to Human Diseases. Frontiers in Chemistry, 2016; 4.
20. Xu, X T, et al. A2B AR activation attenuates acute lung injury by inhibiting alveolar epithelial cell apoptosis both in vivo and in vitro. American Journal of Physiology-Cell Physiology, 2018; 315 (4): C558-C570.
21. Csoka, B, et al. Adenosine promotes alternative macrophage activation via A2A and A2B receptors. FASEB journal: official publication of the Federation of American Societies for Experimental Biology, 2012; 26 (1): 376-86.
22. Koscso, B, et al. Adenosine augments IL-10 production by microglial cells through an A2B adenosine receptor-mediated process. Journal of immunology, 2012; 188 (1): 445-53.
23. Csoka, B, et al. A2B Adenosine Receptors Prevent Insulin Resistance by Inhibiting Adipose Tissue Inflammation via Maintaining Alternative Macrophage Activation. Diabetes, 2014; 63 (3): 850-66.
24. Nemeth, Z H, et al. Adenosine augments IL-10 production by macrophages through an A2B receptor-mediated posttranscriptional mechanism. J Immunol, 2005; 175 (12): 8260-70.
25. Patel, N, et al. A2B Adenosine Receptor Induces Protective Antihelminth Type 2 Immune Responses. Cell Host Microbe, 2014; 15 (3): 339-50.
26 Hasko, G, et al. Adenosine inhibits IL-12 and TNF-[alpha] production via adenosine A2a receptor-dependent and independent mechanisms. Faseb J, 2000; 14 (13): 2065-74.
27. Csoka, B, et al. A2A adenosine receptors and C/EBPbeta are crucially required for IL-10 production by macrophages exposed to Escherichia coli. Blood, 2007; 110 (7): 2685-95.
28. Csoka, B, et al. Adenosine A2A receptor activation inhibits T helper 1 and T helper 2 cell development and effector function. FASEB J, 2008; 22 (10): 3491-9.
29. Hasko, G, et al. Adenosine receptor agonists differentially regulate IL-10, TNF-alpha, and nitric oxide production in RAW 264.7 macrophages and in endotoxemic mice. J Immunol, 1996; 157 (10): 4634-40.
30. Hasko, G, et al. An agonist of adenosine A3 receptors decreases interleukin-12 and interferon-gamma production and prevents lethality in endotoxemic mice. European journal of pharmacology, 1998; 358 (3): 261-8.
31. Szabo, C, et al. Suppression of macrophage inflammatory protein (MIP)-1alpha production and collagen-induced arthritis by adenosine receptor agonists. Br J Pharmacol, 1998; 125 (2): 379-87.
32. Kelestemur, T, et al. A 2a Adenosine Receptors Regulate Multiple Organ Failure after Hemorrhagic Shock in Mice. Shock, 2022; 58 (4): 321-331.
33. Enjyoji, K, et al. Targeted disruption of cd39/ATP diphosphohydrolase results in disordered hemostasis and thromboregulation. Nat Med, 1999; 5 (9): 1010-7.
34 Mizumoto, N, et al. CD39 is the dominant Langerhans cell associated ecto-NTPDase: Modulatory roles in inflammation and immune responsiveness. Nature Medicine, 2002; 8(4): 358-365.
35 Eltzschig, H K, et al. Central role of Sp1-regulated CD39 in hypoxia/ischemia protection. Blood, 2009; 113 (1): 224-32.
36. Eltzschig, H K, et al. Endogenous adenosine produced during hypoxia attenuates neutrophil accumulation: coordination by extracellular nucleotide metabolism. Blood, 2004; 104 (13): 3986-92.
37. Grenz, A, et al. Contribution of E-NTPDasel (CD39) to renal protection from ischemia-reperfusion injury. FASEB J, 2007; 21 (11): 2863-73.
38. Hart, M L, et al. SP1-dependent induction of CD39 facilitates hepatic ischemic preconditioning. J Immunol, 2010; 184 (7): 4017-24.
39. Hotchkiss, R S and I E Karl The pathophysiology and treatment of sepsis. N Engl J Med, 2003; 348 (2): 138-50.
40. Kohler, D, et al. CD39/ectonucleoside triphosphate diphosphohydrolase 1 provides myocardial protection during cardiac ischemia/reperfusion injury. Circulation, 2007; 116(16): 1784-94.
41. R. O. Leite, POdS, C. B. Haas, F. da Silveira, K. R. Mohr, A. P. S. Bertoni, M. S. Soares, J. H. Azambuja, M. Dal Prá, L. L. P. da Cruz, N. E. Gelsleichter, K. Begnini, G. Hasko, M. R. Wink, R. M. Spanevello & E. Braganhol ATPergic signaling disruption in human sepsis as a potential source of biomarkers for clinical use. Clinical and Experimental Medicine, 2023.
42. Riedemann, N C, et al. The enigma of sepsis. J Clin Invest, 2003; 112(4): 460-7.
43. Sayegh, M N, et al. Hydrogel delivery of purinergic enzymes improves cardiac ischemia/reperfusion injury. J Mol Cell Cardiol, 2023; 176:98-109.
44. Hart, M L, et al. Role of extracellular nucleotide phosphohydrolysis in intestinal ischemia-reperfusion injury. FASEB J, 2008; 22 (8): 2784-97.
45. Handa, M and G Guidotti Purification and cloning of a soluble ATP-diphosphohydrolase (apyrase) from potato tubers (Solanum tuberosum). Biochem Biophys Res Commun, 1996; 218(3): 916-23.
46. Thompson, L F, et al. Crucial role for ecto-5′-nucleotidase (CD73) in vascular leakage during hypoxia. J Exp Med, 2004; 200(11): 1395-405.
47. Lennon, P F, et al. Neutrophil-derived 5′-adenosine monophosphate promotes endothelial barrier function via CD73-mediated conversion to adenosine and endothelial A2B receptor activation. J Exp Med, 1998; 188(8): 1433-43.
48. Eltzschig, H K, et al. Coordinated adenine nucleotide phosphohydrolysis and nucleoside signaling in posthypoxic endothelium: role of ectonucleotidases and adenosine A2B receptors. J Exp Med, 2003; 198(5): 783-96.
49. Synnestvedt, K, et al. Ecto-5′-nucleotidase (CD73) regulation by hypoxia-inducible factor-1 mediates permeability changes in intestinal epithelia. J Clin Invest, 2002; 110(7): 993-1002.
50. Beavis, P A, et al. CD73: a potent suppressor of antitumor immune responses. Trends in Immunology, 2012; 33(5): 231-237.
51. Regateiro, F S, et al. Generation of anti-inflammatory adenosine by leukocytes is regulated by TGF-beta. European Journal of Immunology, 2011; 41(10): 2955-2965.
52. Hasko, G, et al. Ecto-5′-nucleotidase (CD73) decreases mortality and organ injury in sepsis. J Immunol, 2011; 187(8): 4256-67.
53. Zhao, N, et al. Adenosine receptor A2B mediates alcoholic hepatitis by regulating cAMP levels and the NF-κB pathway. Toxicol Lett, 2022; 359:84-95.
54. Tavares, L P, et al. Blame the signaling: Role of CAMP for the resolution of inflammation. Pharmacol Res, 2020; 159:105030.
55. Csoka, B, et al. A2B adenosine receptors protect against sepsis-induced mortality by dampening excessive inflammation. J Immunol, 2010; 185(1): 542-50.
56. Beker, M C, et al. Phosphodiesterase 10A Is a Critical Target for Neuroprotection in a Mouse Model of Ischemic Stroke. Molecular Neurobiology, 2022; 59(1): 574-589.
57. Matute-Bello, G, et al. An official American Thoracic Society workshop report: features and measurements of experimental acute lung injury in animals. Am J Respir Cell Mol Biol, 2011; 44(5): 725-38.

1. A method for treating or reducing development of acute respiratory distress syndrome in a subject, comprising administering to the subject an amount of a CD39 mimic and/or CD73 mimic effective to treat or reduce development of acute respiratory distress syndrome; or
a method of treating hemorrhagic shock or hypovolemic shock in a subject, of reducing the likelihood of multi-organ failure in a subject who has had a hemorrhagic shock or hypovolemic shock, or for ameliorating ischemia-reperfusion injury in an organ of a subject who has had a hemorrhagic shock or hypovolemic shock,
the method comprising administering to the subject an amount of a CD39 mimic and/or CD73 mimic effective to treat hemorrhagic shock or hypovolemic shock; or
a method of decreasing liver injury, or reducing an increase in lung permeability and neutrophil infiltration, associated with hypovolemic shock in a subject comprising transfusing into the subject having hypovolemic shock an amount of a CD39 mimic and/or CD73 mimic, sufficient to decrease liver injury, or sufficient to decrease an increase in lung permeability and neutrophil infiltration, associated with hypovolemic shock.

2. A method comprising:
diagnosing a subject as having hemorrhagic shock or hypovolemic shock, and optionally diagnosing the patient as not having an infection or as not having a non-sterile shock;
administering to the subject so-diagnosed an amount of a CD39 mimic and/or CD73 mimic effective to reduce or treat an effect of hemorrhagic shock or hypovolemic shock.

3. The method of claim 1, wherein the subject has had a physical trauma-induced hemorrhagic shock.

4. The method of claim 1, wherein the subject has a sterile shock or does not have sepsis, septic shock, or shock associated with an infection.

5. The method of claim 4, wherein the amount of a CD39 mimic and/or CD73 mimic are administered as a composition comprising the amount of CD39 mimic and/or CD73 mimic and blood or a blood product.

6. The method of claim 5, wherein the method is for treating hemorrhagic shock or hypovolemic shock in a subject.

7. The method of claim 5, wherein the method is for ameliorating ischemia-reperfusion injury in an organ of a subject who has had a hemorrhagic shock or hypovolemic shock.

8. The method of claim 7, wherein the organ is a lung, heart, liver or kidney.

9. The method of claim 1, wherein the amount of CD39 mimic and/or CD73 mimic is administered systemically and/or by transfusion.

10. The method of claim 1, wherein no adenosine receptor ligand(s) are administered to the subject.

11. The method of treating hypovolemic shock in a subject of claim 1, comprising transfusing into the subject having hypovolemic shock an amount of a blood product or blood replacement product which comprises an amount of a CD39 mimic and/or CD73 mimic, sufficient to treat hypovolemic shock.

12. The method of claim 11, wherein the blood product or blood replacement product comprises packed red blood cells, whole blood, plasma or platelets.

13. The method of claim 1, further comprising identifying the subject as requiring a blood transfusion prior to administration of the CD39 mimic and/or CD73 mimic, and administering the CD39 mimic and/or CD73 mimic in the form of a blood transfusion or blood product transfusion to the subject so-identified.

14. The method of claim 13, wherein the CD39 mimic and/or CD73 mimic is/are soluble.

15. A composition for treating hemorrhagic shock or hypovolemic shock, or for ameliorating ischemia-reperfusion injury in an organ, comprising:
a blood or blood product; and
an amount of a CD39 mimic and/or CD73 mimic.

16. The composition of claim 15, wherein the CD39 mimic is recombinant human apyrase.

17. The composition of claim 15, wherein the CD39 mimic is recombinant human CD39.

18. The composition of claim 15, wherein the CD73 mimic is recombinant human CD73.

19. The composition of claim 15, wherein the blood or blood product comprises packed red blood cells, whole blood, plasma or platelets.
